(19) United States
(12) Patent Application Publication
(10) Pub. No.: US 2023/0131870 A1
(43) Pub. Date: Apr. 27, 2023

(54) SEQUESTERING CARBON AND MANAGING AND RESTORING PLANT HEALTH IN PROBLEM SOILS

(71) Applicant: Paul Skinner, St. Helena (CA)

(72) Inventor: Paul Skinner, St. Helena (CA)

(21) Appl. No.: 17/967,439

(22) Filed: Oct. 17, 2022

Related U.S. Application Data
(60) Provisional application No. 63/270,313, filed on Oct. 21, 2021.

(51) Int. Cl. C05D 9/00; G01N 33/24; C05G 5/16

(52) U.S. Cl. CPC C05D 9/00; G01N 33/24; C05G 5/16; G01N 2033/245

(57) ABSTRACT

Implementations generally relate to a system for sequestering carbon. In some implementations, a system includes a carbon source, where the carbon source causes soil chemical reactions that enhance nutrient uptake of at least one plant and that increase above and below ground biomass production, and where the carbon source further increases carbon sequestration in soil surrounding roots of the at least one plant. The system also includes a membrane structure configured to expose the roots of the at least one plant to the carbon source.

### CROSS REFERENCE TO RELATED APPLICATIONS

This application claims priority from U.S. Provisional Patent Application No. 63/270,313, entitled “CARBON APPLICATION/SEQUESTRATION SYSTEM,” filed Oct. 21, 2021 (Attorney Docket No. 021028-000400US), which is hereby incorporated by reference as if set forth in full in this application for all purposes.

### BACKGROUND

Climate change associated with rising levels of carbon dioxide (CO₂) requires dramatic and innovative ways to reduce its negative effects on the environment. Agricultural practices have been identified that can help to reduce atmospheric CO₂ levels, but they have been shown to not be significant enough to offset CO₂ production in a major way.

### SUMMARY

Implementations generally relate to a system for sequestering carbon. In some implementations, a system includes a carbon source, where the carbon source causes soil chemical reactions that enhance nutrient uptake of at least one plant and that increase above and below ground biomass production, and where the carbon source further increases carbon sequestration in soil surrounding roots of the at least one plant. The system also includes a membrane structure configured to expose the roots of the at least one plant to the carbon source.

With further regard to the system, in some implementations, the membrane structure is configured to position the carbon source in the soil at a predetermined proximity to the roots of the at least one plant. In some implementations, to expose the roots of the at least one plant to the carbon source, the membrane structure is configured to contain the carbon source, and where the membrane structure is further configured to surround a portion of the roots of the at least one plant. In some implementations, the membrane structure is configured to position portions of the carbon source at predetermined locations within the membrane structure. In some implementations, the carbon source is contained in carbon packets, where the membrane structure is configured to position the carbon packets at fixed positions in the soil surrounding at least a portion of the roots of the at least one plant. In some implementations, the membrane structure includes membrane structure pockets, and where at least some of the membrane structure pockets are configured to contain one or more mineral nutrient treatments. In some implementations, the membrane structure includes membrane structure pockets, and where at least some of the membrane structure pockets are configured to contain one or more biological treatments.

In some implementations, an apparatus includes a carbon source, where the carbon source causes soil chemical reactions that enhance nutrient uptake of at least one plant and that increase above and below ground biomass production, and where the carbon source further increases carbon sequestration in soil surrounding roots of the at least one plant; and a membrane structure configured to expose the roots of the at least one plant to the carbon source.

With further regard to the apparatus, in some implementations, the membrane structure is configured to position the carbon source in the soil at a predetermined proximity to the roots of the at least one plant. In some implementations, to expose the roots of the at least one plant to the carbon source, the membrane structure is configured to contain the carbon source, and where the membrane structure is further configured to surround a portion of the roots of the at least one plant. In some implementations, the membrane structure is configured to position portions of the carbon source at predetermined locations within the membrane structure. In some implementations, the carbon source is contained in carbon packets, where the membrane structure is configured to position the carbon packets at fixed positions in the soil surrounding at least a portion of the roots of the at least one plant. In some implementations, the membrane structure includes membrane structure pockets, and where at least some of the membrane structure pockets are configured to contain one or more mineral nutrient treatments. In some implementations, the membrane structure includes membrane structure pockets, and where at least some of the membrane structure pockets are configured to contain one or more biological treatments.

In some implementations, a computer-implemented method includes providing a carbon source, where the carbon source causes soil chemical reactions that enhance nutrient uptake of at least one plant and that increase above and below ground biomass production, and where the carbon source further increases carbon sequestration in soil surrounding roots of the at least one plant. The method further includes providing a membrane structure configured to expose the roots of the at least one plant to the carbon source. The method further includes measuring one or more of mineral nutrient treatment levels, biological treatment levels, soil characteristics, and a presence of pathogenic fungi or bacteria in the soil surrounding the roots of the at least one plant.

With further regard to the method, in some implementations, the method further includes: providing one or more sensor units; and measuring one or more of the mineral nutrient treatment levels, the biological treatment levels, the soil characteristics, and the presence of pathogenic fungi or bacteria using the one or more sensor units. In some implementations, the membrane structure is configured to position the carbon source in the soil at a predetermined proximity to the roots of the at least one plant. In some implementations, to expose the roots of the at least one plant to the carbon source, the membrane structure is configured to contain the carbon source, and where the membrane structure is further configured to surround a portion of the roots of the at least one plant. In some implementations, the membrane structure is configured to position portions of the carbon source at predetermined locations within the membrane structure. In some implementations, the carbon source is contained in carbon packets, where the membrane structure is configured to position the carbon packets at fixed positions in the soil surrounding at least a portion of the roots of the at least one plant.

A further understanding of the nature and the advantages of particular implementations disclosed herein may be realized by reference of the remaining portions of the specification and the attached drawings.

### BRIEF DESCRIPTION OF THE DRAWINGS

FIG. 1 is a diagram of an example carbon sequestration environment, which may be used for implementations described herein.

FIG. 2 is a diagram of an example membrane structure, including mineral nutrient treatments and biological treatments, according to some implementations.

FIG. 3 is a diagram of an example portion of a membrane structure, including a membrane structure pocket, according to some implementations.

FIG. 4 is an example flow diagram for sequestering carbon, according to some implementations.

FIG. 5 is a block diagram of an example network environment, which may be used for some implementations described herein.

FIG. 6 is a block diagram of an example computer system, which may be used for some implementations described herein.

### DETAILED DESCRIPTION

Implementations described herein enable, facilitate, and manage the plant nutrient requirements of soil and the surrounding growth environment based on a carbon application/sequestration.

In various implementations, a carbon application/sequestration system includes a carbon source, where the carbon source causes soil chemical reactions that enhance nutrient uptake of at least one plant and that increase above and below ground biomass production. Also, the carbon source further increases carbon sequestration in soil surrounding roots of the at least one plant. In various implementations, the system also includes a membrane structure configured to expose the roots of the at least one plant to the carbon source. In various implementations, the membrane structure has pockets that hold mineral nutrient treatments such as carbon in the form of biochar, as well as biological treatments. The membrane structure is configured to contain the carbon source and to surround the roots of a plant such as a tree, which exposes the roots to the carbon source. As used herein, the “membrane structure” may include any flexible, stiff or solid fabric or structure that can be used to hold, transport, position, sequester or otherwise apply a material such as carbon to adjacent soil. Further example implementations of the membrane structure are described in more detail herein.

In some regions of the world, there are serpentine derived soils, which are classified as being toxic. For example, there are approximately 1 million acres of serpentine derived soils in California, which are classified as being toxic in USDA Soil Survey Publications for Lake County and other northern California counties.

In addition, there are numerous disturbed soils resulting from mining, overgrazing, and wildfires that might also be adapted to the carbon application and sequestration system described herein. Serpentine soil's major negative aspect with regard to plant growth and biomass production is an imbalance of calcium to magnesium. This imbalance has combined effects on soil mineralogy and weathering, water infiltration and release, and soil mineral nutrition, which can all reduce plant growth. Disturbed soils can have nutrient imbalances as well as low organic matter and toxic ion concentrations, which exceed many plants' normal ranges. The end result of these soil problems is that plants living on these soils have a very low net annual biomass production compared to plants on nearby soils whose calcium to magnesium ratios are normal, or which do not have the characteristics listed for disturbed soils. There are certain plants that have adapted to serpentine soils and do grow and produce biomass. However, the rate at which they do so is severely reduced.

Many agricultural soils that have become infected by different pathogenic fungi and bacteria, which not only limit the productivity of the plants growing on them but in some cases (e.g., phytophthora, oak root fungus, vine decline fungi) can kill the plants. Implementations described herein may be used where infected soil is replaced with non-infected soil that is isolated by a membrane structure, which may restore plant health, productivity, and longevity while increasing carbon sequestration above and below ground on otherwise severely restricted soils. Similar membrane-attached mineral nutrient and biological additions on pathogen infected soils may improve plant health and may sequester an increased amount of carbon than non-treated soils.

Implementations described herein, include techniques for changing the chemistry of soils such as serpentine soils. Implementations provide a membrane structure that enables the addition of specific additive amendment materials including carbon and various other mineral nutrient treatments, as well as various biological treatments. The addition of such treatments over time thereby alters the mineralogical makeup of the soil chemistry and rebalances particular mineral ratios such as the calcium/magnesium ratio. Implementations accelerate the rebalancing of particular mineral ratios by releasing certain potentially harmful minerals such as magnesium (Mg) from soil cation exchange sites. Reducing certain minerals such as magnesium enables treated soil to store one or more particular groups of nutrients, including cations. An increase in positively charged ions (cations), other than Mg, improves plant nutrition in serpentine soils.

The modification of the chemistry of serpentine or disturbed soils through the carbon application/sequestration system described herein extends the root zone volume for plant water storage and nutrient uptake, produces more biomass, and also sequesters carbon that can be exchanged for carbon credits, used as carbon offsets, or for carbon insetting to reduce atmospheric CO₂ levels. Implementations described herein increase the biomass produced for plants specifically selected to grow on serpentine soils over that of native vegetation. Implementations described herein provide a carbon application/sequestration system that sequesters carbon in particular soils such as serpentine soils and in other disturbed lands. As described in more detail herein, in various implementations, the carbon application/sequestration system includes a carbon source, where the carbon source is located in soil that is within a predetermined proximity to roots of a plant, where the carbon source causes favorable soil chemical reactions that enhance nutrient uptake and that increase above and below ground biomass production, and where the carbon source further adds to favorable carbon sequestration in these soil types.

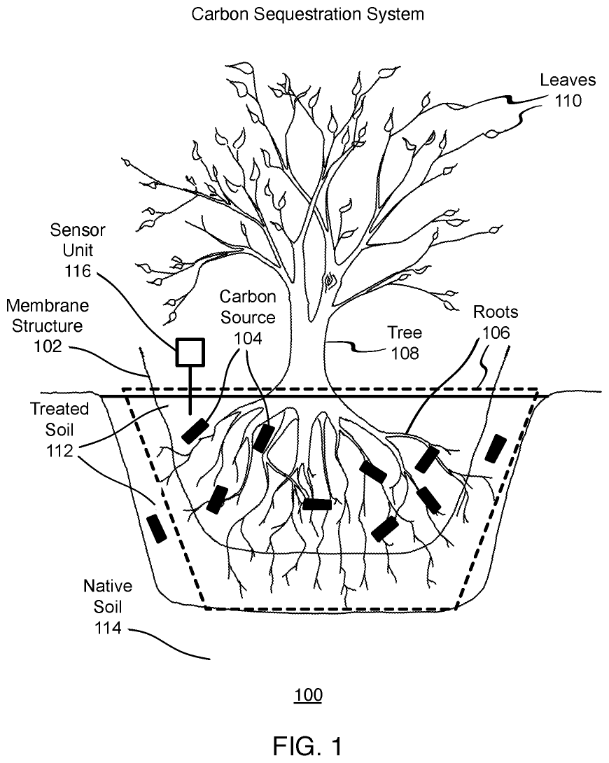

FIG. 1 is a diagram of an example carbon sequestration environment 100, which may be used for implementations described herein. In various implementations, a carbon application/sequestration system in carbon sequestration environment 100 includes a membrane structure 102 that contains a carbon source 104. Carbon source 102 is represented by black bars shown in FIG. 1. The actual size, shape, and form of carbon source 102 may vary, depending on the particular implementation. Membrane structure 102 also surrounds roots 106 (also indicated or demarcated generally by dashed lines) of a plant such as a tree 108 with leaves 110. Membrane structure 102 contains treated soil 112. Native soil 114 surrounds membrane structure 102 and treated soil 112. Also shown is a sensor unit 116. Example implementations directed to sensor unit 116 are described in more detail below.

In various implementations, treated soil 112 is treated with carbon source 104, as well as with other mineral nutrient treatments. Treated soil 112 may also be treated with biological treatments. Example implementations directed to various mineral nutrient treatments and biological treatments are described in more detail below.

While membrane structure 102 contains carbon source 104, in various implementations, membrane structure 102 is semi-porous in order to enable some of the carbon source to permeate the outside of membrane structure 102. Example implementations directed to the semi-porous nature of membrane structure 102 is described in more detail below.

As indicated above, treated soil 112 may contain mineral nutrient treatments and biological treatments, where the mineral nutrient treatments contain carbon source 104. In various scenarios, roots 106 of tree 108 may be bagged in membrane structure 102 in the field, at a nursery, or any other suitable locations. In some implementations, carbon source may also be placed outside of membrane structure.

As described in more detail herein, the semi-porous nature of membrane structure 102 also enables roots 106 of tree 108 to grow through membrane structure 102 to the exterior of membrane structure 102. As the girth of such roots passing through pores of membrane structure 102 grow wider, portions of membrane structure 102 become more porous. As such, more of carbon source 104 may permeate membrane structure 102 in order to treat or further treat the soil outside of membrane structure 102. Carbon source 104 may eventually treat portions native soil 114 depending on the volume of carbon source 104 available in membrane structure 102 and other factors (e.g., membrane structure permeability, water flow, etc.).

In the various implementations described herein, the carbon application/sequestration system enables more CO₂ from the atmosphere to go into the ground. Plants do this naturally. Plant roots also exude CO₂ into the soil, which increases the carbon affecting soil chemical parameters that further benefit plant growth. The carbon application/sequestration system facilitates or enhances this process with the application of carbon source 104 provided by membrane structure 102.

In various implementations, carbon source 104 causes soil chemical reactions that enhance nutrient uptake of at least one plant and that increase above and below ground biomass production. Carbon source 104 also increases carbon sequestration in soil surrounding roots of the at least one plant. In various implementations, the membrane structure is configured to expose the roots of the at least one plant to the carbon source.

The carbon application/sequestration system is especially beneficial when applied to trees, especially bigger trees. More trees provide more biomass to store more carbon in the soil. The amount of biomass generated may be used for carbon-offset credits. Trees may be used to purchase offsets for 20-30 years, for example.

While some implementations are described herein in the context of a single tree whose roots are contained in a single membrane structure, other configurations that involve multiple trees are possible. For example, in some implementations, there may be multiple trees where each tree has a single membrane structure that surrounds its roots. In some implementations, there may be multiple trees in a single large membrane structure that that surrounds the roots of the multiple trees. Alternatively, in some implementations, the carbon application/sequestration system may sequester carbon for one or more trees, yet without a membrane structure. For example, the carbon source may be placed in stand-alone vessels in the soil surrounding the roots of the trees. The carbon source may be place both inside a membrane structure and outside a membrane structure. Benefits of placing the carbon source inside of the membrane structure include control of the location of portions of the carbon source, control over how portions of the carbon source are applied to roots of plants or trees, as wells as an ability to accurately monitor carbon levels as well levels of other treatments both mineral and biological.

While various implementations are described herein in the context of a tree 108, these implementations may apply to other types of plants (e.g., trees, shrubs, herbs, vines, grasses, ferns, mosses, etc.). An advantage of the carbon application/sequestration system being applied to trees is that trees, especially if large and numerous, absorb more CO₂ from the atmosphere smaller plants. The carbon application/sequestration system increases the biomass produced for plants specifically selected to grow in particular soils such as serpentine soils over that of native vegetation.

In various implementations, membrane structure 102 is configured to position carbon source 104 in the soil (e.g., treated soil 112) at a predetermined proximity to roots 106 of at least one plant such as tree 108 in environment 100. The proximity may vary, depending on the particular implementation. For example, the proximity may be based on the size or volume of membrane structure 102.

In various implementations, to expose the roots of the at least one plant to the carbon source, membrane structure 102 is configured to contain carbon source 104 and configured to surround a portion of the roots of at least one plant such as tree 108. By containing carbon source 104 and by surrounding at least a portion of roots 106 of tree 108, roots 106 are exposed to carbon source 104.

Some soils may be classified as non-productive in that they will not grow economically acceptable levels of biomass. For example, serpentine soil does not support a wide range of biomass producing plant species. This may result from chemical imbalances in the soil, for example. In various implementations described herein, treating the soil with carbon improves soil chemical parameters by adjusting the soil pH, which effects the nutrient availability of most plant required nutrients. As described in more detail herein, the carbon source may be mixed with other treatments. The carbon source may also be mixed with native soil (e.g., native serpentine soil, etc.).

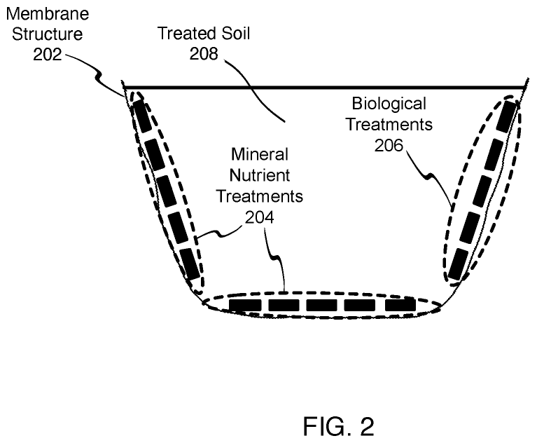

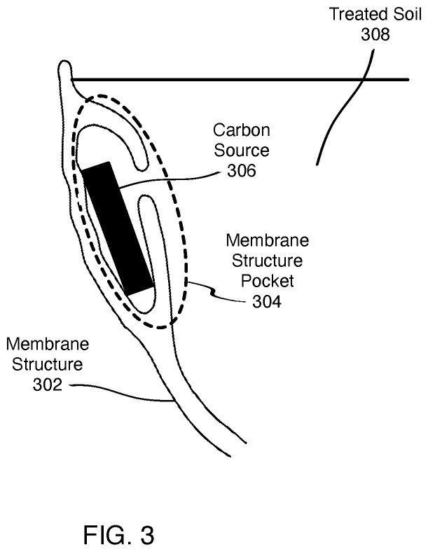

In various implementations, membrane structure 102 may be semi-porous in order to enable carbon from carbon source 104 to permeate membrane structure 102. This enables some roots outside of membrane structure 102 to be exposed to carbon source 104. In some implementations, membrane structure 102 may be more porous in order to allow for more permeation of carbon in the case of fast-growing roots, where it may be desirable for roots that grow through and extend well beyond the boundary of membrane structure 102 to still be exposed to carbon source 104. In this scenario, a higher volume of the carbon source may be initially contained by membrane structure 102 in order for the carbon source to last longer. In some implementations, membrane structure 102 may be less porous in order to allow for less permeation of carbon in the case of slow-growing roots, where it may be desirable to contain more carbon within the boundary of membrane structure 102 so that the carbon lasts longer. Further example implementations directed to the semi-porous nature of membrane structure 102 are described in more detail below in connection with FIG. 2 and FIG. 3, for example.

Regardless of the permeability, the system may include a sensor unit 116 to monitor carbon and nitrogen levels as well as other levels (e.g., levels of mineral nutrient treatments, biological treatments, soil physical and chemical parameters etc.). If the level of carbon or other treatments in environment 100 falls below predetermined threshold level(s), the system may generate an alert to prompt a replenishing of carbon or other treatments.

In other implementations, carbon sequestration environment 100 may not have all of the components shown and/or may have other elements including other types of elements instead of, or in addition to, those shown herein. For example, in various implementations, sensor unit 116 may communicate with a client device (not shown) associated with the carbon application/sequestration system 100 for monitoring the levels of mineral nutrient treatments and biological treatments. The number of sensor units may vary, depending on the implementation. As described in more detail herein, each sensor unit 116 may monitor one or more different types of soil treatments.

In various implementations, as indicated above, the carbon source causes favorable soil chemical reactions. For example, favorable soil chemical reactions may be reactions that enhance nutrient uptake. Other favorable soil chemical reactions may be reactions that increase above and below ground biomass production. Carbon source 104 further adds favorable carbon sequestration in various soil types. Sensor use 116 may facilitate in maintaining carbon levels above a desired level. Soils with between 2% and 4% organic matter contain approximately 1.16-2.32% organic carbon. In some implementations, sensors that measure CO₂ respiration may be used to measure the biological activity of micro-organisms associated with organic matter breakdown.

In various implementations, the carbon application/sequestration system utilizes plant materials that have been either been identified or produced specifically to enhance carbon sequestration on serpentine and/or disturbed soils through genetic manipulation and/or traditional plant breeding and hybridization techniques. In various implementations, the carbon application/sequestration system may utilize various mineral nutrient treatments, including a carbon source and biological treatments including beneficial mycorrhizae fungi to improve the health of soils. In various implementations, portions of carbon source 104 are exposed to soil surrounding roots 106 contained within membrane structure 102 and exposed to soil surrounding roots outside and in close proximity to membrane structure 102. Treated soil 112 refers to soil that is exposed to carbon source 104. Native soil 114 refers to soil that is not exposed to or at least not currently exposed to the carbon source or other mineral nutrient treatments, and not exposed to biological treatments.

FIG. 2 is a diagram of an example membrane structure 202, including mineral nutrient treatments 204 and biological treatments 206, according to some implementations. Shown is membrane structure 202. Mineral nutrient treatments 204 and biological treatments 206 are indicated with dashed lines, as shown in FIG. 2. In various implementations, membrane structure 202 is semi-porous to allow water and some particles of treated soil including carbon to permeate the soil outside of membrane structure 202. Membrane structure 202 is configured to contain mineral nutrient treatments 204 and biological treatments 206. As indicated herein, mineral nutrient treatments 204 include the carbon source (e.g., in the form of biochar). Membrane structure 202 is configured to contain treated soil 208 (e.g., soil treated by mineral nutrient treatments 204 and/or biological treatments 206, etc.).

The carbon source may be a discrete carbon source or a self-contained carbon source in that the carbon source may be separate and distinct from other forms (e.g., mixtures, compounds, etc.). In some implementations, the carbon source may be pure carbon. In some implementations, the carbon source may be in the form of a chemical mixture containing carbon, or a carbon compound such as CO₂, organic material containing composts, or other materials that react to emit carbon. The carbon source may be mixed with soil alone, with compost, or with other types of treatments, which improves the soil chemistry.

In various implementations, mineral nutrient treatments 204 may include the carbon source (e.g., in the form of biochar). The mineral nutrient treatments may also include complexing agents for sodium (Na) and magnesium (Mg), etc., as well as other treatment sources such as nitrogen (N), phosphorus (P), and potassium (K), etc. In some implementations, mineral nutrient treatments may also include cation and anion exchange agents such as zeolites, etc. Biological treatments may include biostimulant capsules, composts, crab shells, root hormones, calcium, etc., all of which improve the soil health.

In some implementations, membrane structure 202 may be a semi-porous container such as a semi-porous bag. The semi-porous container may be made of fiber or a suitable material or materials that enable membrane structure 202 to be semi-porous. The particular fiber or material may vary, depending on the particular implementation. For example, in various implementations, jute and/or industrial hemp or other organic materials may be used for membrane structure 202.

Membrane structure 202 may include any flexible, stiff or solid fabric or structure that may be used to hold, transport, position, sequester or otherwise apply a material such as carbon to adjacent soil. In various implementations, membrane structure 202 functions to release or disseminate the material to the adjacent soil. One way to achieve this is for membrane structure 202 to degrade over time due to elemental or environmental effects (e.g., dissolving, etc.). Natural or synthetic (e.g., plastic, etc.) fabrics may be used. In various implementations, membrane structure 202 may be designed to release the material at an intended time, over an intended time interval, at a desired concentration, or based on other criteria. Other variations of membrane structure 202 are possible.

In some implementations, membrane structure 202 may be made of a biodegradable fiber. In some implementations, membrane structure 202 may be a food-safe fiber as this could be applied to growing animal feed or food crops that may also help to generate carbon credits.

In various implementations, membrane structure 202 is configured to position portions of the carbon source at predetermined locations within membrane structure 202. These locations may position at least some of the carbon source at a fixed proximity to the root portion or roots of a tree (not shown). For example, as shown, attached to membrane structure 202 are mineral nutrient treatments 204, where mineral nutrient treatments 204 include carbon (e.g., in form of biochar). Also attached to membrane structure 202 are biological treatments 206. These mineral nutrient treatments 204 and biological treatments 206 are attached to membrane structure 202 such that they are positioned along the inner surface of membrane structure 202. While the carbon source may be initially applied at a fixed proximity from the roots of a plant or tree, the proximity will change as the roots grow and expand toward the boundary of membrane structure 202 and potentially through pores of membrane structure 202. The initial proximity is based on an amount of carbon exposure to the roots desired, which will change over time.

In various implementations, the carbon source may be contained in carbon packets. For example, mineral nutrient treatments 204 may include carbon in the form of biochar, where the biochar is contained in packets. These packets may be referred to as biochar packets or carbon packets. As shown, membrane structure 202 may be configured to position the carbon packets at fixed positions in the soil surrounding at least a portion of the roots of at least one plant. Positioning the carbon packets or other mineral nutrient treatments or biological treatments at fixed positions may be achieved using membrane structure pockets on membrane structure 202. Example implementations directed to such membrane structure pockets are described in detail below in connection with FIG. 2, for example.

A cross-section of membrane structure 202 is shown. Mineral nutrient treatments 204 and biological treatments 206 are represented by black bars on the inner surface of membrane structure 202 and also indicated by dashed lines, as shown. In various implementations, mineral nutrient treatments 204 and biological treatments 206 may be distributed in multiple directions and spread across the entire inner surface of membrane structure 202. In this implementation, mineral nutrient treatments 204 and biological treatments 206 maintain a fixed positions relative to roots of the root portion of the tree.

In various implementations, mineral nutrient treatments 204 include carbon as well as other mineral nutrient treatments described herein. For example, mineral nutrient treatments 204 may include cation and anion exchange agents such as zeolites, biochar, etc. Biological treatments 206 may include mineral nutrient treatments described herein, as well as biostimulants, rooting hormones, etc.

In various implementations, the mineral nutrient treatments 204 and biological treatments 206 may be arranged randomly or arranged in a predefined pattern on membrane structure 202. By being spread out across different locations on membrane structure 202, mineral nutrient treatments 204 and biological treatments 206 are exposed to more soil thereby treating more of the soil.

For ease of illustration, mineral nutrient treatments 204 are shown as grouped together and biological treatments 206 are shown as grouped together. The groupings, however, may vary depending on the particular implementation. For example, mineral nutrient treatments 204 and biological treatments 206 may be grouped together in close proximity to each other. Also, the locations of mineral nutrient treatments 204 and biological treatments 206 are not limited to being attached in some manner to membrane structure 202. For example, mineral nutrient treatments 204 and biological treatments 206 may be mixed with soil at various different locations within membrane structure 202 including yet not limited to being attached to membrane structure 202.

FIG. 3 is a diagram of an example portion of a membrane structure 302, including a membrane structure pocket 304, according to some implementations. In various implementations, membrane structure 302 has membrane structure pockets. For ease of illustration, one membrane structure pocket 302 is shown. There may be numerous membrane structure pockets and the number may vary, depending on the particular implementation. At least some of the membrane structure pockets are configured to contain one or more mineral nutrient treatments. Carbon source 306 is an example of a type of mineral nutrient treatment. Similarly, at least some of the membrane structure pockets are configured to contain one or more biological treatments.

The form of carbon source 306 may vary. For example, the carbon sources such as lime and gypsum may be powder or capsules, compressed into pellets, bars, or spikes. The carbon source in any of these forms and other forms may be placed in membrane structure pockets such as membrane structure pocket 304. In some implementations, the carbon source may be mixed with soil outside of member structure pockets.

Shown is a cross section of a portion of a membrane structure 302 and membrane structure pocket 304. A carbon source 306 is contained in membrane structure pocket 304. For ease of illustration, one membrane structure pocket is shown. In operation, there may be numerous membrane structure pockets attached to membrane structure 302. Membrane structure 302 is configured to contain treated soil 308 (e.g., soil treated by carbon source 306, etc.). Although implementations disclosed herein are described in the context of a tree and associated root portion, the implementations may also apply to various types of shrubs, vines, and other plants.

In various implementations, membrane structure pocket 304 is a pocket with an opening for inserting carbon source 306 or other mineral nutrient treatments, or for inserting one or more of biological treatments (not shown), or combination thereof. Inserting such treatments in the different membrane structure pockets of membrane structure 302 enables the spreading of treatments, including carbon source 306, in treated soil 308 contained in membrane structure 302.

In some implementations, membrane structure pocket 304 may be integrated with membrane structure 302, as shown. The carbon source may be manufactured into the membrane structure pockets or manually placed in the membrane structure pockets. In some implementations, some membrane structure pockets may be removably attached to membrane structure 302. This enables tailored distribution of carbon source 306 in the soil. This also enables an increase or decrease in the number of membrane structure pockets attached to a given membrane structure. Such a change in the number of membrane structure pockets enables a proportional change in the amount of the carbon source and/or other mineral nutrient treatments and biological treatments to be exposed to the soil. For example, more membrane structure pockets increase the rate of application of one or more of the mineral nutrient treatments and/or biological treatments as more membrane structure pockets are filled with such treatments.

In various implementations, some membrane structure pockets may have openings on the inside of the membrane structure. Some membrane structure pockets may have openings on the outside of the membrane structure. In some implementations, some membrane structure pockets may be stand-alone pockets that are attachable and detachable to the inside or outside of membrane structure. As such, the membrane structure enables the carbon source to be positioned within the membrane structure to treat soil both within the membrane structure and outside of the membrane structure. In some implementations, some membrane structure pockets may be added to the soil detached from the membrane structure.

As indicated above, in various implementations, membrane structure 302 enables carbon from the carbon source 306 to permeate membrane structure 302. Membrane structure 302 being semi-porous allows water and treated soil including carbon to permeate the soil (e.g., native soil) outside of the membrane structure. In some implementations, the membrane structure may have sufficiently large pores to enable small roots to penetrate the membrane structure and to extend to the outside of the membrane structure.

As water moves through the carbon source, carbon is released into the native soil contained within the membrane structure to treat that native soil. In various implementations, water also washes carbon material through the membrane structure into native soil outside of the membrane structure to treat that native soil.

In various implementations, the material used for the membrane structure pockets may break down over time to enable the carbon source to permeate and treat more soil, thereby increasing the overall volume of treated soil. In various implementations, membrane pockets may be enclosed and become more porous and/or decay and/or dissolve over time when exposed to water.

The change in permeability of the membrane structure may be controlled. In some implementations, the membrane structure may allow treatments and roots to pass through to the outside of the membrane structure. In various implementations, the membrane structure becomes more porous as roots that permeate the membrane structure grow bigger. This increases the rate of application of one or more of the mineral nutrient treatments and/or biological treatments. In some implementations, the membrane structure is made from a fiber material that stretches from roots growing bigger and pushing against the fiber material. In some implementations, the membrane structure is made from a fiber material that tears from roots growing bigger and pushing against the fiber material. With these various techniques, more carbon from carbon sources permeates the membrane structure and treats soil outside of the membrane structure over time as roots of the tree grow and expand out and down.

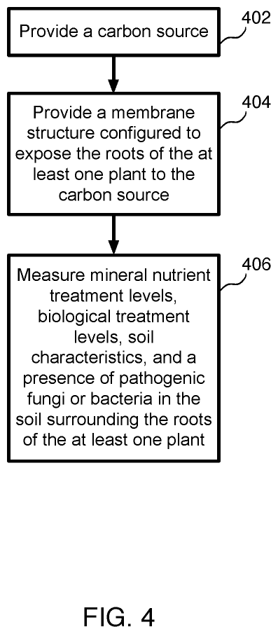

FIG. 4 is an example flow diagram for sequestering carbon, according to some implementations. In various implementations, a method is initiated at block 402, where a system provides a carbon source. As indicated herein, the carbon source causes soil chemical reactions that enhance nutrient uptake of at least one plant and that increase above and below ground biomass production. Also, the carbon source further increases carbon sequestration in soil surrounding roots of the at least one plant.

At block 404, the system provides a membrane structure configured to expose the roots of the at least one plant to the carbon source. As indicated herein, in various implementations, the membrane structure is configured to position the carbon source in the soil at a predetermined proximity to the roots of the at least one plant. The membrane structure is also configured to contain the carbon source and to surround a portion of the roots of the at least one plant. The membrane structure is also configured to position portions of the carbon source at predetermined locations within the membrane structure. In various implementations, the carbon source is contained in carbon packets, where the membrane structure is configured to position the carbon packets at fixed positions in soil surrounding at least a portion of the roots of the at least one plant.

At block 406, the system measures mineral nutrient treatment levels and/or biological treatment levels and/or soil characteristics and/or a presence of pathogenic fungi or bacteria in the soil surrounding the roots of the at least one plant. In various implementations, the system provides one or more sensor units. The system measures the mineral nutrient treatment levels and/or the biological treatment levels and/or the soil characteristics and/or the presence of pathogenic fungi or bacteria using the one or more sensor units. In various implementations, the system measures these levels in the soil, and detects if the mineral nutrient treatment levels, including carbon levels and/or the biological treatment levels, fall below a predetermined threshold. In some implementations, the system may generate an alert if the mineral nutrient treatment levels including carbon levels and/or the biological treatment levels fall below a predetermined threshold. In various implementations, the system measures various soil characteristics including pH, temperature, soil moisture content or tension. In various implementations, if the system measures any presence of pathogenic fungi or bacteria and/or a level of pathogenic fungi or bacteria above a predetermined threshold, the system may generate an alert.

The following describes various implementations directed to sensor units for measuring mineral nutrient treatment levels and/or biological treatment levels in the soil. As indicated above in connection with FIG. 1, in various implementations, the system includes one or more sensor units such as sensor unit 116 of FIG. 1, for example. The carbon sequestration system may have any number of sensor units. Also, some sensor units may be positioned above the soil surface as shown in the example implementation shown in FIG. 1. Some sensor units may also be positioned underneath the soil.

In various implementations, each sensor unit measures one or more of mineral nutrient treatment levels biological treatment levels, and different soil environmental, physical, or chemical characteristics. In these various implementations, each sensor unit may have one or more probes having one or more lengths in order to measure treatment levels at different portions of the soil.

The carbon source may break down and reduce the amount of some types of chemicals to improve the health of the soil. A given sensor unit may detect if and when the amount of a given chemical drops below a predetermined threshold. Also, the carbon source may increase the amount of some types of chemicals to improve the plant nutrient requirements of the soil. A sensor unit may detect if and when the amount of a given chemical increases above a predetermined threshold.

In various implementations, the carbon sequestration system may provide an alarm if and when the sensor unit detects the levels of one or more mineral nutrient treatments and/or biological treatments falling below or rising above one or more respective predetermined threshold levels. The trigger for the alarm may vary depending on the particular implementation. For example, a user might want to know if the level of a particular treatment decreases to a desired level or increases to an undesired level that might be harmful. Conversely, a user might want to know if the level of a helpful treatment decreases to an undesired level that is harmful or increases to a desired level. For example, adding a calcium rich nutrient source may raise the pH of acid soils (e.g., pH 4.5 to 6.0) to a pH between 6-7. If the pH over time falls below a pH of 5.5, or exceeds a pH of 8.0, certain mineral nutrients become less available to plant roots and therefore more calcium or other acidifying agent additions may be warranted.

In various implementations, each sensor is configured to measure levels of one or more different types of treatments. In some implementations, a given sensor of the group of sensors may be configured to be dedicated to measure the level of a particular type of treatments. For example, one or more given sensor units may be dedicated to measure the effects of increased carbon levels on soil chemical parameters associated with increased plant growth.

In various implementations, a given sensor may be triggered if a given sensor measures the level of a given type of treatment and the associated soil chemical parameter level falls below a predetermined threshold level. In some implementations, the predetermined threshold level may be an absolute level. In some implementations, the predetermined threshold level may be based on an absolute level and the volume of soil in the membrane structure.

Implementations described herein provide various benefits. For example, implementations improve the health of soil in and around the membrane structure of the carbon sequestration system. Implementations described herein also provide an alarm if and when the levels of one or more mineral nutrient treatments and/or biological treatments fall below one or more respective predetermined threshold levels.

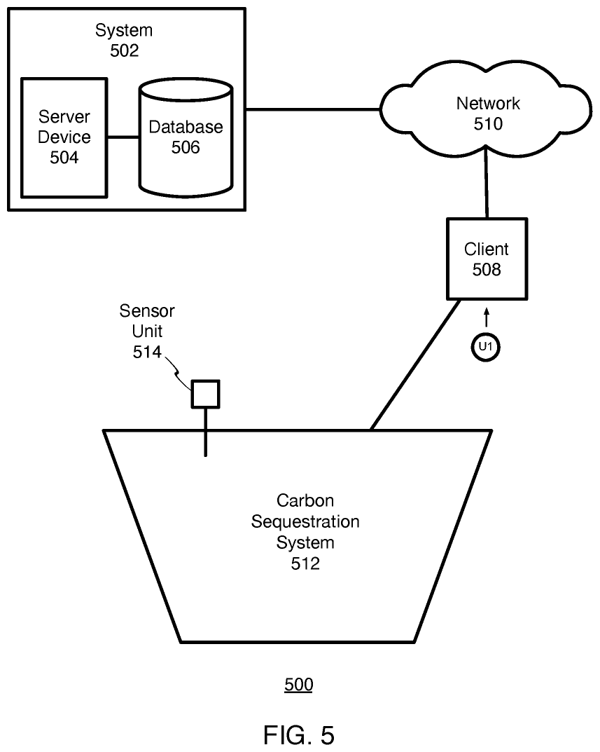

FIG. 5 is a block diagram of an example network environment 500, which may be used for some implementations described herein. In some implementations, network environment 500 includes a system 502, which includes a server device 504 and a database 506. System 502 may be used to perform implementations described herein. Network environment 500 also includes a client device 508, which may communicate with system 502 via network 510. Network 510 may be any suitable communication network such as a Wi-Fi network, Bluetooth network, the Internet, etc., or combination thereof.

Network environment 500 also includes a carbon application/sequestration system 512, which may be used to implement the carbon application/sequestration system in carbon sequestration environment 100 of FIG. 1. Network environment 500 also includes a sensor unit 514 for measuring levels of mineral nutrient treatments including carbon and biological treatments. In various implementations, system 502 may be used to perform operations described herein. System 502 may be used to monitor and store levels of mineral nutrient treatments and biological treatments, as well as to generate alerts if any given levels of mineral nutrient treatments or biological treatments deviate from desired levels.

For ease of illustration, FIG. 5 shows one block for each of system 502, server device 504, network database 506, client device 508, network 510, carbon sequestration system 512, and sensor unit 514. Blocks 502, 504, 506, 508, 510, 512, and 514 may represent multiple systems, server devices, network databases, client devices, networks, carbon sequestration systems, and sensor units. In other implementations, environment 500 may not have all of the components shown and/or may have other elements including other types of elements instead of, or in addition to, those shown herein.

While server device 504 of system 502 performs implementations described herein, in other implementations, any suitable component or combination of components associated with system 502 or any suitable processor or processors associated with system 502 may facilitate performing the implementations described herein.

In the various implementations described herein, a processor of system 502 and/or a processor of client device 508 cause the elements described herein (e.g., soil information, mineral nutrient treatment levels, biological treatment levels, etc.) to be displayed in a user interface on one or more display screens.

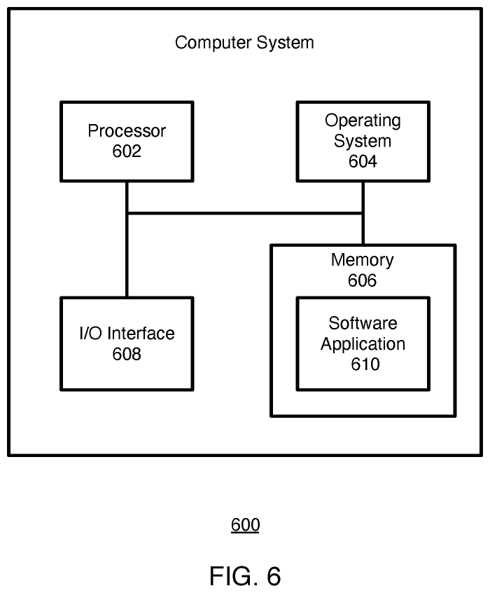

FIG. 6 is a block diagram of an example computer system 600, which may be used for some implementations described herein. For example, computer system 600 may be used to implement server device 504 of FIG. 5 and/or other systems associated with the carbon application/sequestration system of carbon sequestration environment 100 of FIG. 1, as well as to perform implementations described herein. In some implementations, computer system 600 may include a processor 602, an operating system 604, a memory 606, and an input/output (I/O) interface 608. In various implementations, processor 602 may be used to implement various functions and features described herein, as well as to perform the method implementations described herein. While processor 602 is described as performing implementations described herein, any suitable component or combination of components of computer system 600 or any suitable processor or processors associated with computer system 600 or any suitable system may perform the steps described. Implementations described herein may be carried out on a user device, on a server, or a combination of both.

Computer system 600 also includes a software application 610, which may be stored on memory 606 or on any other suitable storage location or computer-readable medium. Software application 610 provides instructions that enable processor 602 to perform the implementations described herein and other functions. Software application may also include an engine such as a network engine for performing various functions associated with one or more networks and network communications. The components of computer system 600 may be implemented by one or more processors or any combination of hardware devices, as well as any combination of hardware, software, firmware, etc.

For ease of illustration, FIG. 6 shows one block for each of processor 602, operating system 604, memory 606, I/O interface 608, and software application 610. These blocks 602, 604, 606, 608, and 610 may represent multiple processors, operating systems, memories, I/O interfaces, and software applications. In various implementations, computer system 600 may not have all of the components shown and/or may have other elements including other types of components instead of, or in addition to, those shown herein.

A “processor” may include any suitable hardware and/or software system, mechanism, or component that processes data, signals or other information. A processor may include a system with a general-purpose central processing unit, multiple processing units, dedicated circuitry for achieving functionality, or other systems. Processing need not be limited to a geographic location, or have temporal limitations. For example, a processor may perform its functions in “real-time,” “offline,” in a “batch mode,” etc. Portions of processing may be performed at different times and at different locations, by different (or the same) processing systems. A computer may be any processor in communication with a memory. The memory may be any suitable data storage, memory and/or non-transitory computer-readable storage medium, including electronic storage devices such as random-access memory (RAM), read-only memory (ROM), magnetic storage device (hard disk drive or the like), flash, optical storage device (CD, DVD or the like), magnetic or optical disk, or other tangible media suitable for storing instructions (e.g., program or software instructions) for execution by the processor. For example, a tangible medium such as a hardware storage device can be used to store the control logic, which can include executable instructions. The instructions can also be contained in, and provided as, an electronic signal, for example in the form of software as a service (SaaS) delivered from a server (e.g., a distributed system and/or a cloud computing system).

Although the description has been described with respect to particular implementations thereof, these particular implementations are merely illustrative, and not restrictive. Concepts illustrated in the examples may be applied to other examples and implementations.

It will also be appreciated that one or more of the elements depicted in the drawings/figures can also be implemented in a more separated or integrated manner, or even removed or rendered as inoperable in certain cases, as is useful in accordance with a particular application. It is also within the spirit and scope to implement a program or code that can be stored in a machine-readable medium to permit a computer to perform any of the methods described above.

As used in the description herein and throughout the claims that follow, “a”, “an”, and “the” includes plural references unless the context clearly dictates otherwise. Also, as used in the description herein and throughout the claims that follow, the meaning of “in” includes “in” and “on” unless the context clearly dictates otherwise.

Thus, while particular implementations have been described herein, latitudes of modification, various changes, and substitutions are intended in the foregoing disclosures, and it will be appreciated that in some instances some features of particular implementations will be employed without a corresponding use of other features without departing from the scope and spirit as set forth. Therefore, many modifications may be made to adapt a particular situation or material to the essential scope and spirit.

What is claimed is:

1. A system comprising:
a carbon source, wherein the carbon source causes soil chemical reactions that enhance nutrient uptake of at least one plant and that increase above and below ground biomass production, and wherein the carbon source further increases carbon sequestration in soil surrounding roots of the at least one plant; and
a membrane structure configured to expose the roots of the at least one plant to the carbon source.

2. The system of claim 1, wherein the membrane structure is configured to position the carbon source in the soil at a predetermined proximity to the roots of the at least one plant.

3. The system of claim 1, wherein, to expose the roots of the at least one plant to the carbon source, the membrane structure is configured to contain the carbon source, and wherein the membrane structure is further configured to surround a portion of the roots of the at least one plant.

4. The system of claim 1, wherein the membrane structure is configured to position portions of the carbon source at predetermined locations within the membrane structure.

5. The system of claim 1, wherein the carbon source is contained in carbon packets, wherein the membrane structure is configured to position the carbon packets at fixed positions in the soil surrounding at least a portion of the roots of the at least one plant.

6. The system of claim 1, wherein the membrane structure comprises membrane structure pockets, and wherein at least some of the membrane structure pockets are configured to contain one or more mineral nutrient treatments.

7. The system of claim 1, wherein the membrane structure comprises membrane structure pockets, and wherein at least some of the membrane structure pockets are configured to contain one or more biological treatments.

8. An apparatus comprising:
a carbon source, wherein the carbon source causes soil chemical reactions that enhance nutrient uptake of at least one plant and that increase above and below ground biomass production, and wherein the carbon source further increases carbon sequestration in soil surrounding roots of the at least one plant; and
a membrane structure configured to expose the roots of the at least one plant to the carbon source.

9. The apparatus of claim 8, wherein the membrane structure is configured to position the carbon source in the soil at a predetermined proximity to the roots of the at least one plant.

10. The apparatus of claim 8, wherein, to expose the roots of the at least one plant to the carbon source, the membrane structure is configured to contain the carbon source, and wherein the membrane structure is further configured to surround a portion of the roots of the at least one plant.

11. The apparatus of claim 8, wherein the membrane structure is configured to position portions of the carbon source at predetermined locations within the membrane structure.

12. The apparatus of claim 8, wherein the carbon source is contained in carbon packets, wherein the membrane structure is configured to position the carbon packets at fixed positions in the soil surrounding at least a portion of the roots of the at least one plant.

13. The apparatus of claim 8, wherein the membrane structure comprises membrane structure pockets, and wherein at least some of the membrane structure pockets are configured to contain one or more mineral nutrient treatments.

14. The apparatus of claim 8, wherein the membrane structure comprises membrane structure pockets, and wherein at least some of the membrane structure pockets are configured to contain one or more biological treatments.

15. A computer-implemented method comprising:
providing a carbon source, wherein the carbon source causes soil chemical reactions that enhance nutrient uptake of at least one plant and that increase above and below ground biomass production, and wherein the carbon source further increases carbon sequestration in soil surrounding roots of the at least one plant;
providing a membrane structure configured to expose the roots of the at least one plant to the carbon source; and
measuring one or more of mineral nutrient treatment levels, biological treatment levels, soil characteristics, and a presence of pathogenic fungi or bacteria in the soil surrounding the roots of at least one plant.

16. The method of claim 15, further comprising:
providing one or more sensor units; and
measuring one or more of the mineral nutrient treatment levels, the biological treatment levels, the soil characteristics, and the presence of pathogenic fungi or bacteria using the one or more sensor units.

17. The method of claim 15, wherein the membrane structure is configured to position the carbon source in the soil at a predetermined proximity to the roots of the at least one plant.

18. The method of claim 15, wherein, to expose the roots of the at least one plant to the carbon source, the membrane structure is configured to contain the carbon source, and wherein the membrane structure is further configured to surround a portion of the roots of the at least one plant.

19. The method of claim 15, wherein the membrane structure is configured to position portions of the carbon source at predetermined locations within the membrane structure.

20. The method of claim 15, wherein the carbon source is contained in carbon packets, wherein the membrane structure is configured to position the carbon packets at fixed positions in the soil surrounding at least a portion of the roots of the at least one plant.
